(19) United States
(12) Patent Application Publication
(10) Pub. No.: US 2020/0030574 A1
(43) Pub. Date: Jan. 30, 2020

(54) Multilumen Curved Split-tip Catheter

(71) Applicant: Mohamed A Basiony, Kenmore, WA (US)

(72) Inventor: Mohamed A Basiony, Kenmore, WA (US)

(21) Appl. No.: 16/045,461

(22) Filed: Jul. 25, 2018

(51) Int. Cl. A61M 25/00

(52) U.S. Cl. CPC A61M 25/0071; A61M 25/007; A61M 25/003; A61M 25/0662; A61M 25/0074; A61M 2205/0205; A61M 25/0097

(57) ABSTRACT

A multilumen curved split-tip catheter comprises a proximal end, a distal end and an elongated portion arranged between a proximal end and a distal end, defining a longitudinal axis. Elongated portion includes triple lumens for a blood flow therethrough, two arterial lumens and one venous lumen. The triple lumens are fixed to make a venous lumen is located between two arterial lumens along the length of the catheter to two dividing points. Also, the triple lumens are not at the same level with respect to each other inside a blood vessel after insertion. Each lumen extends between a distal end and a proximal end and includes a port at a distal end thereof in a communication with the lumen. Distal end has three curved distal segments that are splitted (separated) from each other with respect to a longitudinal axis.

### BACKGROUND

In hemodialysis application wherein, a blood is withdrawal from a blood vessel for treatment by an artificial kidney device and the treated blood is introduced back into a blood vessel.

Various known multilumen curved split-tip catheters have been employed, a typical example of a multilumen curved split-tip catheter is a dual lumen catheter, to withdrawal a blood from a blood vessel through one lumen of the catheter (arterial lumen), then the dialyzed blood is returned to the patient through a second lumen of the catheter (venous lumen). Venous lumen is typically longer than arterial lumen to reduce a blood recirculation in a forward blood lines configuration but in a reverse blood lines configuration, the recirculation may be more than 20 percentage.

Multilumen curved split-tip catheter was designed to automatically center the catheters ports within a blood vessel to reduce a fibrin sheath formation, thrombosis and vessel wall occlusions by keeping the tips of the catheter away from the blood vessel wall.

The drawbacks of a dual lumen curved split-tip catheters may be a blood recirculation especially in a reverse blood lines configuration and a moderate blood flow.

It would be beneficial to provide a multilumen curved split-tip catheters that may reduce a recirculation in a reverse blood lines configuration and may maximize amount of blood that is taken during a hemodialysis process.

### SUMMARY

Accordingly, a hemodialysis multilumen curved split-tip catheter is described to may address the above issues.

The catheter may have a curved configuration to utilize the benefits of self-centric curved split-tip catheters. The catheter may have a proximal end, a distal end and an elongated portion arranged between a proximal end and a distal end, defining a longitudinal axis. Elongated portion may include triple lumens for a blood flow therethrough, two arterial lumens for extraction of untreated blood and one venous lumen (third lumen) for introducing a treated blood back to a blood vessel.

Each lumen may extend between a distal end and a proximal end and may include a port at a distal end thereof in a communication with the lumen. The triple lumens may be fixed to make a venous lumen located between two arterial lumens alone the length of the catheter to two dividing points that may be fixed in a location at a distal end of the catheter.

Distal end may have three distal segments that may splitted (separated) from each other with respect to a longitudinal axis at two dividing points at a proximal end of said three distal segments.

Proximal end of the catheter may attach to a hub portion with suture wings assembly, which in turn may be connected to extension tubings. Extension tubings may fluidly connect catheter lumens to a blood treatment unit or a dialysis machine.

### BRIEF DESCRIPTION OF THE DRAWINGS

The accompanying drawings, which are incorporated in, and constitute a part of the specification, illustrate or exemplify implementation of the present disclosure and, together with the description, generally explain the principles and features of the present disclosure. The reader should understand that no limitation to the precise arrangement and instrumentalities shown. Modifications, alternation and further application of the principles of the disclosure are also included in the scope of this disclosure. The drawings are briefly described as follows:

FIG. 1 illustrates a perspective view of a multilumen curved split-tip long term catheter according to the present disclosure.

FIG. 1a is an enlarged cross-sectional view of the elongated portion of the catheter assembly of FIG. 1 taken along line 1a-1a according to the present disclosure.

FIG. 1b is an enlarged cross-sectional view of the distal segments of the catheter assembly of FIG. 1 taken along line 1b-1b according to the present disclosure.

FIG. 2 illustrates a perspective view of a multilumen curved split-tip short term catheter according to the present disclosure.

FIG. 3 is an enlarged perspective view, in section of a hub portion of the catheter of FIG. 1 and FIG. 2 taken along line 3-3 according to the present disclosure.

FIG. 4 is enlarged perspective view of a curved distal configuration of the catheter of FIG. 1 with contact points with a blood vessel wall according to the present disclosure.

FIG. 5 is an enlarged cross-sectional view of the elongated portion of the catheter of FIG. 1 and FIG. 2 with two arterial lumens and one venous lumen according to the present disclosure.

### DETAILED DESCRIPTION

The following detailed description illustrates the principal of the disclosure by way of example not by way of limitation. While a reference use of the present disclosure describes a multilumen curved split-tip catheter to be used in hemodialysis, additional non-limiting usage would also include hemofiltration, hemodifiltration, blood adsorption, apheresis, as those of ordinary skill in the art will readily understand. Also, while a reference use of the present disclosure describes a multilumen curved split-tip catheter to be used in a blood treatment, additional non-limiting usage would also include any fluids.

The hemodialysis multilumen curved split-tip catheter of present disclosure can be utilized as a short term or a long term vascular access for the above treatments and may be made by a biocompatible material like; polyethene, polycarbonate, Silicon or any other suitable material. The catheter may also include an anti-microbial coating such as silver, methylene blue and the like. The catheter may be of any suitable size between 6 to 16 French circumferences, or any other suitable sizes.

The configuration of the catheter may be manipulated to facilitate placement of the catheter into a blood vessel. In one implementation, the catheter may be compressed into a substantially liner profile using a sheath. In another implementation, the catheter may be placed over two guidewires with or without stylet/s and with two self-sealed side holes in a venous lumen (not shown for simplicity) to facilitate the placement of the catheter into a blood vessel.

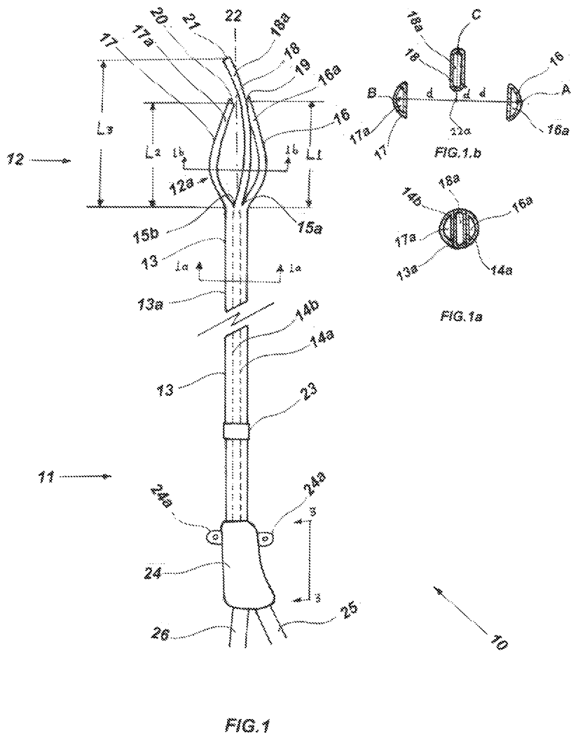

Now referring to FIG. 1, it illustrates a catheter 10 that may comprise an elongated portion 13, a proximal end 11 and a distal end 12, extended longitudinally to form a longitudinal axis 22. Elongated portion 13 may be a straight or may have a pre-curved configuration. Also, elongated portion with an outer diameter 13a may have three lumens 16a, 17a and 18a that may extend distally and proximally along a longitudinal axis 22 and may have two dividing septum's 14a and 14b. Three lumens 16a, 17a and 18a may be two arterial lumens; first lumen (arterial) 16a and a second lumen (arterial) 17a for extraction of untreated blood and a third lumen (venous) 18a for introducing a treated blood back to a blood vessel.

Proximal end 11 of a catheter 10 may have a cuff 23 (that may be polyester felt or any other material) and a hub portion 24 with suture wings 24a, which in turn may be connected to a first extension tube 25 and a second extension tube 26 as is standard in dialysis catheters. A first extension tube 25 and a second extension tube 26 fluidly connect catheter lumens 16a, 17a and 18a to a blood treatment unit or a dialysis machine (not shown for simplicity).

Distal end 12 of a catheter 10 may be splitted (separated) with respect to a longitudinal axis 22 into a first distal segment (arterial) 16, a second distal segment (arterial) 17 and a third distal segment (venous) 18 at dividing points 15a and 15b.

A first distal segment (arterial) 16, a second distal segment (arterial) 17 and a third distal segment (venous) 18, may bend (curved) longitudinally outward with respect to a longitudinal axis 22 to form a curved distal configuration 12a to utilize the benefit of self-centric catheters that are automatically center a first distal segment (arterial) 16, a second distal segment (arterial) 17 and a third distal segment (venous) 18 within a blood vessel to reduce fibrin sheath formation, thrombosis and vessel wall occlusions by keeping a first distal port 19 of a first distal segment (arterial) 16, a second distal port 20 of a second distal segment (arterial) 17 and a third distal port 21 of a third distal segment (venous) 18 of a catheter 10 away from a blood vessel wall.

A first distal segment (arterial) 16 may have a first lumen 16a and a first distal port 19. A second distal segment (arterial) 17 may have a second lumen 17a and a second distal port 20. A third distal segment (venous) 18 may have a third lumen 18a and a third distal port 21. In another implementation, a first distal segment (arterial) 16 may also have a distal tip, a second distal segment (arterial) 17 may also have a distal tip and a third distal segment (venous) 18 may also have a distal tip. Said distal tips do not show for simplicity.

First distal segment (arterial) 16 and second distal segment (arterial) 17 may have each a D-shape or any other shapes in cross section, while a third distal segment (venous) 18 may have approximately a rectangular shape (with curved corners) or any other shape. Elongated portion may have an exterior with generally round or any other shapes in cross section. Also, elongated portion may have an internal longitudinally extending lumen of D-shape for both arterial lumens 16a and 17a and approximately a rectangular shape (with curved corners) for a venous lumen 18a, or any other shapes.

“L1” represents a longitudinal length between a dividing point 15a and a first distal port 19 while “L2” represents a longitudinal length between a dividing point 15b and a second distal port 20. “L1” and “L2” in one implementation may be equal and may be about 25 mm to about 35 mm or any suitable length. In another implementation, “L1” and “L2” may be with a different length. Also, in one implementation a dividing point 15a and a dividing point 15b may be at the same distance with respect to a cuff 23. In another implementation, a dividing point 15a and a dividing point 15b may not be at the same distance with respect to a cuff 23. “L3” represents a longitudinal length between a dividing point 15b and a third distal port 21, it may be about 40 mm to about 60 mm or any suitable length.

FIG. 1a illustrates enlarged cross-sectional view of an elongated portion 13 of a catheter of FIG. 1 taken along line 1a-1a, wherein an outer diameter 13a, a dividing septum 14a, a dividing septum 14b, a first lumen (arterial) 16a, a second lumen (arterial) 17a, and a third lumen (venous) 18a.

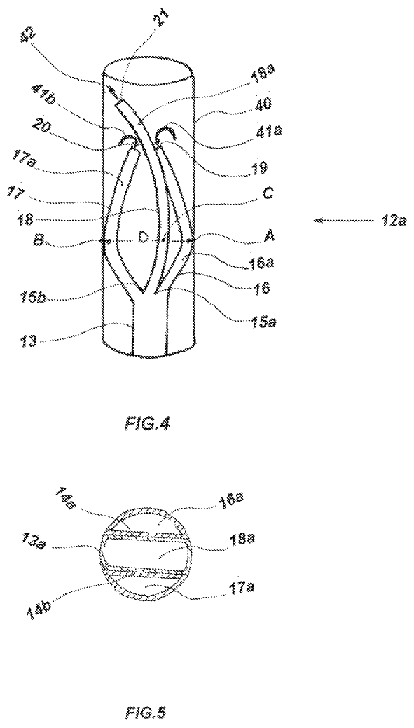

FIG. 1b illustrates enlarged cross-sectional view of distal segments 16, 17 and 18 of a catheter of FIG. 1 taken along line 1b-1b, wherein a first distal segment (arterial) 16 may have a first lumen (arterial) 16a, a second distal segment (arterial) 17 may have a second lumen (arterial) 17a, and a third distal segment (venous) 18 may have a third lumen (venous) 18a. Also, as can be seen in FIG. 1b and FIG. 4, a first distal segment (arterial) 16 and a second distal segment (arterial) 17 may be on a different level with respect to a third distal segment (venous) 18.

Also, in FIG. 1b, “d” represents a distance between a contact point “A” and a longitudinal axis point 22a of a longitudinal axis 22 wherein as in FIG. 4, “A” is a contact point between a first distal segment (arterial) 16 and a side wall of a blood vessel 40. Also, “d” represents a distance between a contact point “B” and a longitudinal axis point 22a of a longitudinal axis 22 wherein as in FIG. 4, “B” is a contact point between a second distal segment (arterial) 17 and a side wall of a blood vessel 40. Also, “d” represents a distance between a contact point “C” and a longitudinal axis point 22a of a longitudinal axis 22 wherein as in FIG. 4, “C” is a contact point between a third distal segment (venous) 18 and an upper wall of a blood vessel 40. So, in one implementation, contact points, “A”, “B” and “C” may be at a same distance “d” from a longitudinal axis point 22a of a longitudinal axis 22. In another implementation, contact points, “A”, “B” and “C” may not be at a same distance “d” from a longitudinal axis point 22a of a longitudinal axis 22.

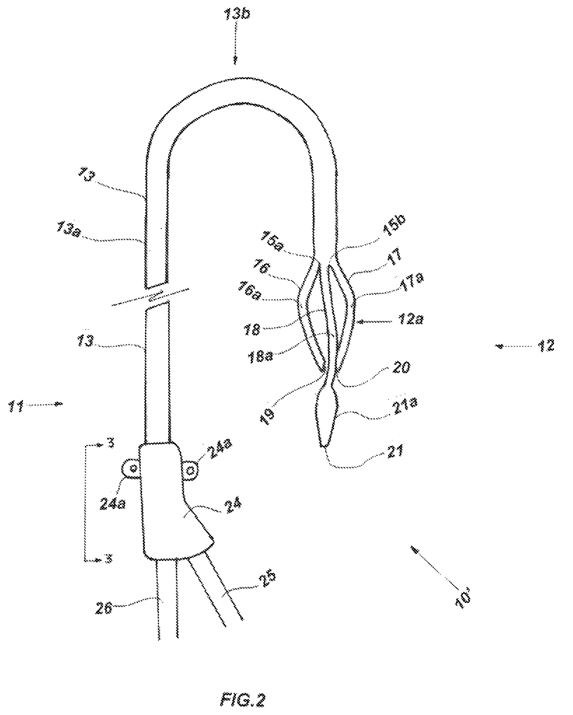

FIG. 2 illustrates a catheter 10′ which is exactly like a catheter 10 except it is a short-term catheter without a cuff 23 and it may have a different material and a tapered third distal port (venous) 21a, for easy insertion. Also FIG. 2 illustrates a pre-curved configuration 13b of an elongated portion 13 while the rest of components of a catheter 10′ are the same as of a catheter 10.

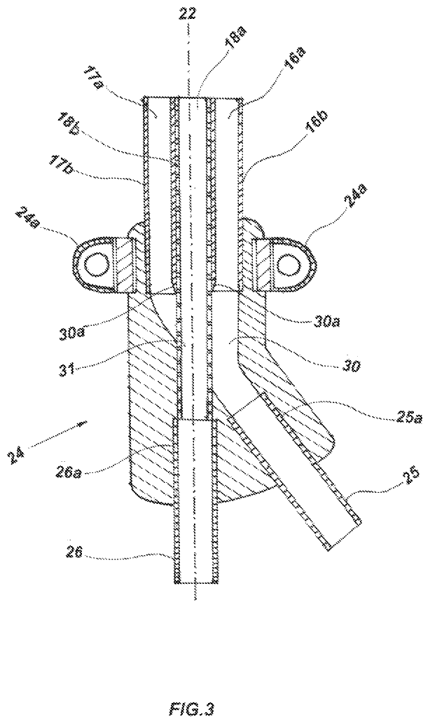

FIG. 3 illustrates an enlarged perspective view, in section of a hub portion 24 of a catheter 10 of FIG. 1 and a catheter 10′ of FIG. 2 taken along line 3-3, wherein a first lumen 16a (arterial) with a first lumen proximal end 16b, a second lumen (arterial) 17a with a second lumen proximal end 17b, a third lumen (venous) 18a with a third lumen proximal end 18b and suture wings 24a to secure a hub portion 24 to a patient after insertion of a catheter 10 and 10′ into a patient.

Arterial passageway 30 of a first lumen (arterial) 16a and a second lumen (arterial) 17a within a hub portion 24 between a first lumen proximal end 16b of a first lumen (arterial) 16a and a second lumen proximal end 17b of a second lumen (arterial) 17a and a first extension tube 25 may bend within a hub portion 24 at an angle of approximately 15 degrees away from a longitudinal axis 22, or any other suitable degrees.

Arterial passageway 30 of a first lumen (arterial) 16a and a second lumen (arterial) 17a may taper to a narrow diameter 30a at a third lumen proximal end 18b of a third lumen (venous) 18a to may provide enhanced flow through arterial passageway 30. Arterial passageway 30 may direct a blood flow therethrough between a first lumen (arterial) 16a and a second lumen (arterial) 17a and a first extension tube 25, while venous passageway 31 may direct a blood flow therethrough between a third lumen (venous) 18a and a second extension tube 26.

A first lumen proximal end 16b of a first lumen (arterial) 16a, a second lumen proximal end 17b of a second lumen (arterial) 17a and a third lumen proximal end 18b of a third lumen (venous) 18a may terminate in a hub portion 24. Inside a hub portion 24, a third lumen (venous) 18a may exit a first lumen (arterial) 16a and a second lumen (arterial) 17a. A first lumen proximal end 16b of a first lumen (arterial) 16a and a second lumen proximal end 17b of a second lumen (arterial) 17a may fluidly communicate with a first extension tube 25 within a hub portion 24. A distal end 25a of a first extension tube 25 may dispose and secure by a hub portion 24. A third lumen proximal end 18b of a third lumen (venous) 18a may fluidly communicate with a second extension tube 26 within a hub portion 24. A distal end 26a of a second extension tube 26 my dispose and secure by a hub portion 24.

FIG. 4 illustrates enlarged view of a curved distal configuration 12a of a catheter 10 within a blood vessel 40, wherein an elongated portion 13, a first distal segment (arterial) 16 with a first lumen (arterial) 16a and a first distal port 19, a second distal segment (arterial) 17 with a second lumen (arterial) 17a and a second distal port 20 and a third distal segment (venous) 18 with a third lumen (venous) 18a and a third distal port 21. The same is applicable to a catheter 10′.

FIG. 4 also illustrates a contact point “A” of a first distal segment (arterial) 16 with a side wall of a blood vessel 40, a contact point “B” of a second distal segment 17 (arterial) with a side wall of a blood vessel 40 and a contact point “C” of a third distal segment (venous) 18 with an upper wall of a blood vessel 40. In another implementation, a contact point “C” of a third distal segment (venous) 18 may be with a lower wall of a blood vessel 40. So, a first distal segment (arterial) 16 and a second distal segment (arterial) 17 may be at the same level while a third distal segment (venous) 18 may be at a higher level or a lower level with respect to each other inside a blood vessel.

In one implementation contact points “A”, “B” and “C” may be at the same distance longitudinally with respect to a cuff 23. In another implementation contact points “A”, “B” and “C” may be at a different distance longitudinally with respect to a cuff 23. “D” represents a distance between contact “A” and contact “B”. A distance “D” may be equal to a double of a distance “d” in FIG. 1b and may be about 10 mm to about 30 mm or any suitable diameter based on the place of insertion of a catheter 10 if it is in a superior or in an inferior vena cava or in peripheral veins. The same is applicable to a catheter 10′.

Those skilled in the art will recognize that contact points “A”,“B” and “C” may automatically center a first distal segment (arterial) 16, a second distal segment (arterial) 17 and a third distal segment (venous) 18 inside a blood vessel 40 to may reduce fibrin sheath formation, thrombosis and vessel wall occlusions by keeping a first distal port 19, a second distal port 20 and a third distal port 21 away from a wall of a blood vessel 40. The same is applicable to a catheter 10′.

Also, FIG. 4 illustrates a blood inlet 41a into a first distal port 19, a blood inlet 41b into a second distal port 20 and a blood outlet 42 from a third distal port 21. Those skilled in the art will recognize that a first lumen (arterial) 16a and a second lumen (arterial) 17a (two lumens) of a catheter 10 and 10′ may maximize the amount of blood that is taken during a hemodialysis process compare to one arterial lumen of a dual lumen catheter. Also, with two arterial lumens 16a and 17a may no need to reverse a blood lines to avoid a blood recirculation (which it may be more than 20 percentage in a reverse blood lines configuration) as it may be with an existing dual lumen curved split-tip catheter.

FIG. 5 illustrates an enlarged cross-sectional view of elongated portion 13 of catheter 10 and a catheter 10′ wherein, a third lumen (venous) 18a may be located between a first lumen (arterial) 16a and a second lumen (arterial) 17a. Also, FIG. 5 illustrates outer diameter 13a, a dividing septum 14a and a dividing septum 14b.

1. A multilumen curved split-tip catheter comprising:
a proximal end, a distal end and an elongated portion arranged between a proximal end and a distal end, defining a lonitudinal aixs;
an elonagted portion includes triple lumens for a blood flow therethrough, two arterial lumens and one venous lumen. Said triple lumens are fixed to make a venous lumen is located between two arterial lumens along the length of the catheter to two dividing points. Each of said lumen extends between said distal end and said a proxmal end and includes a port at a distal end thereof in a communication with said lumen;
a distal end comprising three curved splitted distal segments that are splitted with respect to a longitudinal axis at said two dividing points at a proximal end of said three distal segments which in turn is coupled with a distal end of said elongated portion, and
a proximal end comprising a hub portion with suture wings assembly, which in turn is connected to two extension tubings.

2. A multilumen curved split-tip catheter of claim 1, wherein said elonaged portion has triple lumens.

3. A multilumen curved split-tip catheter of claim 2, wherein said triple lumens are two arterial lumens and one venous lumen.

4. A multilumen curved split-tip catheter of claim 3, wherein said venous lumen is located between said two arterial lumens alonge the length of the catheter to said two dividing points.

5. A multilumen curved split-tip catheter of claim 1, wherein each of said lumen extends between said distal end and said proxmal end and includes a port at a distal end thereof in a communication with said lumen.

6. A multilumen curved split-tip catheter of claim 1, wherein said distal end has three splitted distal segments with respect to a longitudinal axis at said two dividing points.

7. A multilumen curved split-tip catheter of claim 6, wherein siad three splitted distal segements have a curved configuration.

8. A multilumen curved split-tip catheter of claim 6, wherein said three splitted distal segments are not at the same level with respect to each other inside a blood vessel after insertion.

9. A multilumen curved split-tip catheter of claim 8, wherein siad two arterial segments are at a different level with respect to said venous distal segment.

10. A multilumen curved split-tip catheter of claim 6, wherein siad three splitted distal segments have three contact points with a blood vessel wall.

11. A multilumen curved split-tip catheter of claim 10, wherein said three ontact points are at a different level with respect to each other.

12. A multilumen curved split-tip catheter of claim 10, wherein said three contact points are at the same distance with respect to the longitudinal axis point of said longitudinal axis.

13. A multilumen curved split-tip catheter of claim 1, wherein the arterial passageway inside said hub portion directs a blood flow therethrough between said two arterial lumens and said first extension tube.

14. A multilumen curved split-tip catheter of claim 1, wherein the venous passageway inside said hub portion directs a blood flow therethrough between said venous lumen and said second extension tube.

15. A multilumen curved split-tip catheter of claim 13, wherein said arterial passageway bends within said hub portion at an angle away from said longitudinal axis.
